(19) United States
(12) Patent Application Publication
(10) Pub. No.: US 2004/0223916 A1
(43) Pub. Date: Nov. 11, 2004

(54) Canister for a metered dose inhaler

(76) Inventors: Peter Colin Weston Burt, Ware (GB); Laurie Koon-Hung Kwok, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/781,537

(22) Filed: Feb. 18, 2004

Related U.S. Application Data
(60) Provisional application No. 60/448,775, filed on Feb. 20, 2003.

(51) Int. Cl. A61L 9/04

(52) U.S. Cl. 424/45; 128/200.23

(57) ABSTRACT

A canister for a metered dose inhaler having part or all of its internal surfaces coated with one or more fluorocarbon polymers, optionally in combination with one or more non-fluorocarbon polymers, said canister having a wall with a thickness in the range 0.55 mm to 1.00 mm.

### CROSS-REFERENCE TO RELATED APPLICATIONS

The present application claims priority to U.S. Provisional Application No. 60/448,775 filed Feb. 20, 2003, the disclosure of which is incorporated herein by reference in its entirety.

### FIELD OF THE INVENTION

The present invention generally relates to canisters for use in inhalation devices and device systems, inhalation devices and device systems using such canisters, and methods of administering active ingredient(s) using such devices and device systems.

### BACKGROUND OF THE INVENTION

Drugs for treating respiratory and nasal disorders are frequently administered in aerosol formulations through the mouth or nose. One widely used method for dispensing such aerosol drug formulations involves making a suspension formulation of the drug as a finely divided powder in a liquefied gas known as a propellant. The suspension is stored in a sealed container capable of withstanding the pressure required to maintain the propellant as a liquid. The suspension is dispersed by activation of a dose metering valve affixed to the container.

A metering valve may be designed to consistently release a fixed, predetermined mass of the drug formulation upon each activation. As the suspension is forced from the container through the dose metering valve by the high vapour pressure of the propellant, the propellant rapidly vaporizes leaving a fast moving cloud of very fine particles of the drug formulation. This cloud of particles is directed into the nose or mouth of the patient by a channelling device such as a cylinder or open-ended cone. Concurrently with the activation of the aerosol dose metering valve, the patient inhales the drug particles into the lungs or nasal cavity. Systems of dispensing drugs in this way are known as “metered dose inhalers” (MDI's). See Peter Byron, Respiratory Drug Delivery, CRC Press, Boca Raton, Fla. (1990) for a general background on this form of therapy.

Patients often rely on medication delivered by MDI's for rapid treatment of respiratory disorders which are debilitating and in some cases, even life threatening. Therefore, it is essential that the prescribed dose of aerosol medication delivered to the patient consistently meet the specifications claimed by the manufacturer and comply with the requirements of the FDA and other regulatory authorities. That is, every dose in the can must be the same within close tolerances.

Some aerosol drugs tend to adhere to the inner surfaces, i.e., walls of the can, valves, and caps, of the MDI. This can lead to the patient getting significantly less than the prescribed amount of drug upon each activation of the MDI. The problem is particularly acute with hydrofluoroalkane (also known as simply “fluorocarbon”) propellant systems, e.g., P134a and P227, under development in recent years to replace chloroflurocarbons such as P11, P114 and P12.

It has been found that coating the interior can surfaces of MDI's with a fluorocarbon polymer significantly reduces or essentially eliminates the problem of adhesion or deposition of drug on the can walls and thus ensures consistent delivery of medication in aerosol from the MDI as disclosed, for example, in WO96/32099, WO96/32150, WO96/32151, WO96/32345.

However, we have found that canisters coated in fluorocarbon polymers and cured at temperatures, for example, in the range 300-400° C. are annealed and weakened. This results in an increased number of standard canisters, which have a wall thickness of 0.46 to 0.48 mm, ultimately leaking and becoming damaged by, for example, scratching, crushing or denting during manufacture and transportation. These defects are unacceptable and may cause a serious accident if the canister, which is pressurised in use, exploded due to rupturing of the aerosol canister. Additionally canisters annealed in this way are more likely to fail a common industry test for aerosol canisters (ARTA testing), for example, the bases of such canisters may dome and even be blown out by pressure build up within the canister when subjected to said testing. Adjustment to manufacturing equipment cannot alleviate these occurrences.

### SUMMARY OF THE INVENTION

In one aspect, the invention provides a canister for a metered dose inhaler having part or all of its internal surfaces coated with one or more fluorocarbon polymers, optionally in combination with one or more non-fluorocarbon polymers, the canister having a wall with a thickness in the range 0.55 mm to 1.00 mm.

In another aspect, the invention provides a metered dose inhaler including a canister.

In another aspect, the invention provides a metered dose inhaler system including a canister.

In another aspect, the invention provides a method of administering at least one active ingredient to a patient comprising providing a metered dose inhaler; and activating the metered dose inhaler to deliver a pharmaceutically effective amount of the at least one active ingredient to the patient.

These and other aspects are encompassed by the present invention.

### BRIEF DESCRIPTION OF THE DRAWINGS

FIG. 1 shows a perspective view of an indentor. The indentor of FIG. 1 has a scale 1, a weight 2 and in use a can 3 is tested.

FIG. 2 shows a graph of the compression strength profile of annealed aerosol canisters having different wall thicknesses over the first 10 mm compression.

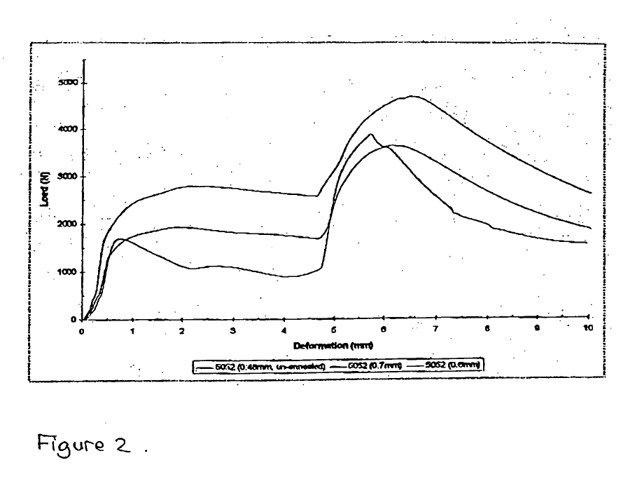

### DETAILED DESCRIPTION OF THE INVENTION

The invention provides a canister for a metered dose inhaler having part or all of its internal surfaces coated with one or more fluorocarbon polymers, optionally in combination with one or more non-fluorocarbon polymers, said canister having a wall with a thickness in the range about 0.55 mm to about 1.00 mm.

Preferably canisters according to the invention have a wall having a thickness in the range 0.55 to 0.70 mm, particularly between 0.55 mm and 0.60 mm, especially about 0.6 mm. Canisters falling within this range have the advantage of adequate strength whilst minimising the amount of material used in their manufacture which results in more efficient use of raw materials, such as aluminium, and ultimately in environmental and coat savings.

About when used in relation to the above ranges includes the values disclosed and variations thereof within engineering tolerances.

In canisters of the present invention the base will generally be approximately the same thickness as the canister walls.

A canister thus comprises a wall and a base.

Wall when used in relation to a canister is a structural component of the canister which optionally in combination with another structural component such as the base and/or another wall defines the internal volume of the canister.

Canisters according to the invention can be stamped or drawn at high speeds and precision by stamping or drawing by known methods. However, the starting thickness of the metal metal sheet must be suitable for ultimately providing a canister with walls of the desired thickness. Preferably canisters according to the invention will be deep drawn.

Preferably the canisters according to the invention are such that they have major external dimensions the same as the external dimensions of standard canisters and therefore can advantageously be used with existing manufacturing lines and accessories such as, valves and actuators. Thus the increased wall thickness in effect reduces the internal dimensions of the canister whilst maintain the external dimensions in most respects unchanged.

Preferably the neck of the canister is be adapted to provide a more gradual transition from the main body of the canister to the orifice where the valve will ultimately sit. This more gradual transfer may be manifest as a reduction in the angle of the slope of the neck from the vertical.

In a further aspect the invention includes a metered dose inhaler comprising a canister according to the invention as described above.

The metered dose inhalers comprising canisters according to the invention are suitable for dispensing an inhalation drug formulation comprising drug, or a physiologically acceptable salt thereof, and a fluorocarbon propellant, optionally in combination with one or more other pharmacologically active agents or one or more excipients.

The term “metered dose inhaler” or “MDI” means a unit comprising a canister, a crimped cap covering the mouth of the canister, and a drug metering valve situated in the cap, while the term “MDI system” also includes a suitable channelling device. The terms “MDI canister” means the container without the cap and valve. The term “drug metering valve” or “MDI valve” refers to a valve and its associated mechanisms which delivers a predetermined amount of drug formulation from an MDI upon each activation. The channelling device may comprise, for example, an actuating device for the valve and a cylindrical or cone-like passage through which medicament may be delivered from the filled MDI can via the MDI valve to the nose or mouth of a patient, e.g. a mouthpiece actuator. The relation of the parts of a typical MDI is illustrated in U.S. Pat. No. 5,261,538 incorporated herein by reference.

The valve typically comprises a valve body having an inlet port through which the pharmaceutical aerosol formulation may enter said valve body, an outlet port through which the pharmaceutical aerosol may exit the valve body and an open/close mechanism by means of which flow through said outlet port is controllable.

The valve may be a slide valve wherein the open/close mechanism comprises a sealing ring and receivable by said sealing ring a valve stem having a dispensing passage, said valve stem being slidably movable within the ring from a valve-closed to a valve-open position in which the interior of the valve body is in communication with the exterior of the valve body via said dispensing passage.

In a preferred aspect, the valve is a metering valve in which the valve body has a metering chamber, a sampling chamber and therebetween a second sealing ring within which the stem is slidably movable, the valve stem having a transfer passage such that in the valve-closed position the dispensing passage is isolated from the metering chamber and the metering chamber is in communication with the sampling chamber via said transfer passage, and in the valve-open position the dispensing passage is in communication with the metering chamber and the transfer passage is isolated from the metering chamber.

Preferably the sealing ring and/or second sealing ring comprises an elastomeric material. The ring is typically resiliently deformable.

The elastomeric material may either comprise a thermoplastic elastomer (TPE) or a thermoset elastomer which may optionally be cross-linked. The sealing ring may also comprise a thermoplastic elastomer blend or alloy in which an elastomeric material is dispersed in a thermoplastic matrix. The elastomers may optionally additionally contain conventional polymer additives such as processing aids, colorants, tackifiers, lubricants, silica, talc, or processing oils such as mineral oil in suitable amounts.

Suitable thermoset rubbers include butyl rubbers, chloro-butyl rubbers, bromo-butyl rubbers, nitrile rubbers, silicone rubbers, fluorosilicone rubbers, fluorocarbon rubbers, polysulphide rubbers, polypropylene oxide rubbers, isoprene rubbers, isoprene-isobutene rubbers, isobutylene rubbers or neoprene (polychloroprene) rubbers.

Suitable thermoplastic elastomers comprise a copolymer of about 80 to about 95 mole percent ethylene and a total of about 5 to about 20 mole percent of one or more comonomers selected from the group consisting of 1-butene, 1-hexene, and 1-octene as known in the art. Two or more such copolymers may be blended together to form a thermoplastic polymer blend.

Another suitable class of thermoplastic elastomers are the styrene-ethylene/butylene-styrene block copolymers. These copolymers may additionally comprise a polyolefin (e.g. polypropylene) and a siloxane.

Preferably, the sealing ring and/or the second sealing ring additionally comprises lubricant material. Suitably, the sealing ring and/or the second sealing ring comprises up to 30%, preferably from 5 to 20% lubricant material.

Preferably, the stem comprises lubricant material. Suitably, the valve stem comprises up to 30%, preferably from 5 to 20% lubricant material.

The term ‘lubricant’ herein means any material which reduces friction between the valve stem and seal. Suitable lubricants include silicone oil or a fluorocarbon polymer such as polytetrafluoroethane (PTFE) or fluoroethylene propylene (FEP).

Lubricant can be applied to the stem, sealing ring or second sealing ring by any suitable process including coating and impregnation, such as by injection or a tamponage process.

Suitable valves are commercially available, for example from Valois SA, France (e.g. DF10, DF30, DF60), Bespak Plc, UK (e.g. BK300, BK356, BK357) and 3M-Neotechnic Ltd UK (e.g. Spraymiser (trade name)).

Typically the valve is sealed to the can by means of a gasket seal. Materials suitable for use in the gasket seal include the elastomeric materials mentioned above as suitable for the sealing ring and/or the second sealing ring.

Valves which are entirely or substantially composed of metal (eg stainless steel) components, save for the seals, (eg Spraymiser, 3M-Neotechnic) are especially preferred for use according to the invention.

Thermoplastic elastomeric material may also be selected from one or more of the following: polyester rubbers, polyurethane rubbers, ethylene vinyl acetate rubber, styrene butadiene rubber, copolyether ester TPE, olefinic TPE, polyester amide TPE and polyether amide TPE. Example TPE materials are described in WO95/02651.

Other suitable elastomers include ethylene propylene diene rubber (EPDM) eg as described in WO92/11190. The EPDM may be present on its own or present as part of a thermoplastic elastomer blend or alloy, e.g. in the form of particles substantially uniformly dispersed in a continuous thermoplastic matrix (e.g. polypropylene or polyethylene). Commercially available thermoplastic elastomer blend and alloys include the SANTOPRENE™ elastomers. Other suitable thermoplastic elastomer blends include butyl-polyethylene (e.g. in a ratio ranging between about 2:3 and about 3:2) and butyl-polypropylene.

The term “fluorocarbon polymers” means a polymer in which one or more of the hydrogen atoms of the hydrocarbon chain have been replaced by fluorine atoms. Thus, “fluorocarbon polymers” include perfluorocarbon, hydrofluorocarbon, chlorofluorocarbon, hydro-chlorofluorocarbon polymers or other halogen substituted derivatives thereof. The “fluorocarbon polymers” may be branched, homo-polymers or co-polymers.

The term “drug formulation” means drug or a physiologically acceptable salt thereof (particularly the sulfate salt) optionally in combination with one or more other pharmacologically active agents such as antiinflammatory agents, analgesic agents or other respiratory drugs and optionally containing one or more excipients. The term “excipients” as used herein means chemical agents having little or no pharmacological activity (for the quantities used) but which enhance the drug formulation or the performance of the MDI system. For example, excipients include but are not limited to surfactants, preservatives, flavorings, antioxidants, antiaggregating agents, and cosolvents, e.g., ethanol and diethyl ether. Drug or salt thereof may be used in the form of its R-isomer.

Suitable surfactants are generally known in the art, for example, those surfactants disclosed in European Patent Application No. 0327777. The amount of surfactant employed is desirable in the range of 0.0001% to 50% weight to weight ratio relative to the drug, in particular, 0.05 to 5% weight to weight ratio. A particularly useful surfactant is 1,2-di[7-(F-hexyl) hexanoyl]-glycero-3-phospho-N,N,N-trimethylethanolamine also known as 3, 5, 9-trioxa-4-phosphadocosan-1-aminium, 17, 17, 18, 18, 19, 19, 20, 20, 21, 21, 22, 22, 22-tridecafluoro-7-[(8, 8, 9, 9, 10, 10, 11, 11, 12, 12,13, 13, 13-tridecafluoro-1-oxotridecyl)oxy]-4-hydroxy-N,N,N-trimethyl-10-oxo-, inner salt, 4-oxide.

A polar cosolvent such as C₂₋₆ aliphatic alcohols and polyols e.g. ethanol, isopropanol and propylene glycol, preferably ethanol, may be included in the drug formulation in the desired amount, either as the only excipient or in addition to other excipients such as surfactants. Suitably, the drug formulation may contain 0.01 to 5% w/w based on the propellant of a polar cosolvent e.g. ethanol, preferably 0.1 to 5% w/w e.g. about 0.1 to 1% w/w.

It will be appreciated by those skilled in the art that the drug formulation for use in the invention may, if desired, contain drug or a salt thereof (e.g. the sulphate) in combination with one or more other pharmacologically active agents. Such medicaments may be selected from any suitable drug useful in inhalation therapy. Appropriate medicaments may thus be selected from, for example, analgesics, e.g. codeine, dihydromorphine, ergotamine, fentanyl or morphine; anginal preparations, e.g. diltiazem; antiallergics, e.g. cromoglycate, ketotifen or nedocromil; antiinfectives e.g. cephalosporins, penicillins, streptomycin, sulphonamides, tetracyclines and pentamidine; antihistamines, e.g. methapyrilene; anti-inflammatories, e.g. beclomethasone (e.g. the dipropionate), flunisolide, budesonide, tipredane or triamcinolone acetonide; antitussives, e.g. noscapine; bronchodilators, e.g. salbutamol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, orciprenaline, or (−)-4-amino-3,5-dichloro-α-[[[6-[2-(2-pyridinyl)ethoxy]hexyl]amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament and/or to minimise the solubility of the medicament in the propellant.

Particularly preferred medicaments for administration using aerosol formulations in accordance with the invention include anti-allergics, bronchodilators and anti-inflammatory steroids of use in the treatment of respiratory disorders such as asthma, COPD or rhinitis by inhalation therapy, for example, cromoglycate (e.g. as sodium salt), albuterol (e.g. as free base or the sulphate), salmeterol (e.g. as xinafoate), formoterol (e.g. as fumarate), terbutaline (e.g. as sulphate), reproterol (e.g. as hydrochloride), a beclomethasone ester (e.g. as dipropionate), a fluticasone ester (e.g. as propionate). Salmeterol, especially salmeterol xinafoate, albuterol sulphate, fluticasone propionate, beclomethasone dipropionate and physiologically acceptable salts and solvates thereof are especially preferred.

It will be appreciated by those skilled in the art that the aerosol formulations according to the invention may, if desired, contain a combination of two or more active ingredients. Thus suitable combinations include bronchodilators (e.g. albuterol or isoprenaline) in combination with an anti-inflammatory steroid (e.g. beclomethasone ester); a bronchodilator in combination with an anti-allergic (e.g. cromoglycate). Exemplary combinations also include: ephedrine and theophylline; fenoterol and ipratropium (e.g. as bromide); isoetharine and phenylephrine; albuterol (e.g. as free base or as sulphate) and beclomethasone ester (e.g. as dipropionate); budesonide and formoterol (e.g. as fumarate) which is of particular interest; and salmeterol (particularly as salmeterol xinafoate) and fluticasone ester (e.g. as propionate) also of particular interest.

“Propellants” used herein mean pharmacologically inert liquids with boiling points from about room temperature (25° C.) to about −25° C. which singly or in combination exert a high vapour pressure at room temperature. Upon activation of the MDI system, the high vapour pressure of the propellant in the MDI forces a metered amount of drug formulation out through the metering valve then the propellant very rapidly vaporizes dispersing the drug particles. The propellants used in the present invention are low boiling fluorocarbons; in particular, 1,1,1,2-tetrafluoroethane also known as “propellant 134a” or “P 134a” and 1,1,1,2,3,3,3-heptafluoropropane also known as “propellant 227” or “P 227”.

Drug formulations for use in the invention may be free or substantially free of formulation excipients e.g. surfactants and cosolvents etc. Such drug formulations are advantageous since they may be substantially taste and odour free, less irritant and less toxic than excipient-containing formulations. Thus, a preferred drug formulation consists essentially of drug or a physiologically acceptable salt thereof, optionally in combination with one or more other pharmacologically active agents particularly salmeterol (e.g. in the form of the xinafoate salt), and a fluorocarbon propellant. Preferred propellants are 1,1,1,2-tetrafluoroethane, 1,1,1,2,3,3,3-heptafluoro-n-propane or mixtures thereof, and especially 1,1,1,2-tetrafluoroethane.

Further drug formulations for use in the invention may be free or substantially free of surfactant. Thus, a further preferred drug formulation comprises or consists essentially of drug (or a physiologically acceptable salt thereof), optionally in combination with one or more other pharmacologically active agents, a fluorocarbon propellant and 0.01 to 5% w/w based on the propellant of a polar cosolvent, which formulation is substantially free of surfactant. Preferred propellants are 1,1,1,2-tetrafluoroethane, 1,1,1,2,3,3,3-heptafluoro-n-propane or mixtures thereof, and especially 1,1,1,2-tetrafluoroethane or 1,1,1,2,3,3,3-heptafluoro-n-propane.

The canisters according to the invention and cap for said canister are made of aluminum or an alloy of aluminum. Preferably the canisters according to the invention are prepared from aluminium, such as aluminium with the alloy number IADS 5052.

Fluorocarbon polymers for use in the invention include fluorocarbon polymers which are made of multiples of one or more of the following monomeric units: tetrafluoroethylene (PTFE), fluorinated ethylene propylene (FEP), perfluoroalkoxyalkane (PFA), ethylene terafluoroethylene (ETFE), vinyldienefluoride (PVDF), and chlorinated ethylene tetrafluoroethylene.

Fluorinated polymers which have a relatively high ratio of fluorine to carbon, such as perfluorocarbon polymers, e.g., PTFE, PFA, and FEP are preferred.

The fluorinated polymer may be blended with non-fluorinated polymers such as polyamides, polyimides, polyethersulfones, polyphenylene sulfides, and amine-formaldehyde thermosetting resins. These added polymers improve adhesion of the polymer coating to the can walls. Preferred polymer blends are PTFE/FEP/polyamideimide, PTFE/polyether sulphone (PES) and FEP-benzoguanamine. Preferably, the fluorocarbon polymers for use in the invention are coated onto MDI cans made of metal, especially MDI cans made of aluminium or an alloy thereof.

Particularly preferred coatings are pure PFA and blends of PTFE and polyethersulphone (PES).

Fluorocarbon polymers are marketed under trademarks such as Teflon®, Tefzel®, Halar® and Hostaflon®, Polyflon® and Neoflon®. Grades of polymer include FEP DuPont 856-200, PFA DuPont 857-200, PTFE-PES DuPont 3200-100, PTFE-FEP-polyamideimide DuPont 856P23485, FEP powder DuPont 532, and PFA Hoechst 6900n. The coating thickness is in the range of about 1 μm to about 1 mm. Suitably the coating thickness is in the range of about 1 μm to about 100 μm, e.g.1 μm to 25 μm. Coatings may be applied in one or more coats

Canisters according to the invention will preferably be completely coated on their internal surface(s).

The invention also includes a process where said canister is coated with a fluorocarbon polymer optionally in combination with a non-fluorocarbon polymer and cured at 300-400° C. such as 380-390° C. and pressure resistant canisters produced by this method.

Canisters according to the invention will preferably contain a pharmaceutical aerosol formulation comprising particulate drug and propellant.

Generally the particle size of the particulate (e.g., micronised) drug should be such as to permit inhalation of substantially all the drug into the lungs upon administration of the aerosol formulation and will thus be less than 100 microns, desirably less than 20 microns, and, in particular, in the range of 1-10 microns, e.g., 1-5 microns.

The final aerosol formulation desirably contains 0.005-10% weight to weight ratio, in particular 0.005-5% weight to weight ratio, especially 0.01-1.0% weight to weight ratio, of drug relative to the total weight of the formulation.

A further aspect of the present invention is a metered dose inhaler comprising a canister according to the invention wherein said metered dose inhaler has part or all of its internal metallic surfaces coated with one or more fluorocarbon polymers, optionally in combination with one or more non-fluorocarbon polymers, for dispensing an inhalation drug formulation comprising drug or a salt thereof and a fluorocarbon propellant optionally in combination with one or more other pharmacologically active agents and one or more excipients.

A particular aspect of the present invention is an MDI comprising a canister according to the invention having essentially all of its internal metallic surfaces coated with PFA or FEP, or blended fluoropolymer resin systems such as PTFE-PES with or without a primer coat of a polyamideimide or polyethersulfone for dispensing a drug formulation defined hereinabove. Preferred drug formulations for use in this MDI consist essentially of drug (or a physiologically acceptable salt thereof, e.g. the sulfate), optionally in combination with one or more other pharmacologically active agents particularly beclomethasone dipropionate (or a solvate thereof), and a fluorocarbon propellant, particularly 1,1,1,2-tetrafluoroethane, 1,1,1,2,3,3,3-heptafluoropropane or mixtures thereof, and especially 1,1,1,2-tetrafluoroethane.

The MDI can may be coated by the means known in the art of metal coating. For example, a metal, such as aluminum, may be precoated as coil stock and cured before being stamped or drawn into the can shape. This method is well suited to high volume production for two reasons. First, the art of coating coil stock well developed and several manufacturers can custom coat metal coil stock to high standards of uniformity and in a wide range of thicknesses. Second, the precoated stock can be stamped or drawn at high speeds and precision by essentially the same methods used to draw or stamp uncoated stock.

Other techniques for obtaining coated cans is by electrostatic dry powder coating or by spraying preformed MDI cans inside with formulations of the coating fluorinated polymer/polymer blend and then curing. The preformed MDI cans may also be dipped in the fluorocarbon polymer/polymer blend coating formulation and cured, thus becoming coated on the inside and out. The fluorocarbon polymer/polymer blend formulation may also be poured inside the MDI cans then drained out leaving the insides with the polymer coat. Conveniently, for ease of manufacture, preformed MDI cans are spray-coated with the fluorinated polymer/polymer blend.

Fluorocarbon polymer film may be blown inside the MDI cans to form bags. A variety of fluorocarbon polymers such as ETFE, FEP, and PTFE are available as film stock.

The appropriate curing temperature is dependent on the fluorocarbon polymer/polymer blend chosen for the coating and the coating method employed. However, for coil coating and spray coating temperatures in excess of the melting point of the polymer are typically required, for example, about 50° C. above the melting point for up to about 20 minutes such as about 5 to 10 minutes eg about 8 minutes or as required. For the above named preferred and particularly preferred fluorocarbon polymer/polymer blends curing temperatures in the range of about 300° C. to about 400° C., e.g. about 350° C. to 380° C. are suitable.

The MDI's taught herein may be prepared by methods of the art (e.g., see Byron, above and U.S. Pat. No. 5,345,980) substituting conventional cans for those coated with a fluorinated polymer/polymer blend. That is, drug or a salt thereof and other components of the formulation are filled into an aerosol can coated with a fluorinated polymer/polymer blend. The can is fitted with a cap assembly which is crimped in place. The suspension of the drug in the fluorocarbon propellant in liquid form may be introduced through the metering valve as taught in U.S. Pat. No. 5,345,980 incorporated herein by reference.

The MDI's with fluorocarbon polymer/polymer blend coated interiors taught herein may be used in medical practice in a similar manner as non-coated MDI's now in clinical use. However the MDI's taught herein are particularly useful for containing and dispensing inhaled drug formulations with hydrofluoroalkane fluorocarbon propellants such as 134a with little, or essentially no excipient and which tend to deposit or cling to the interior walls and parts of the MDI system. In certain cases it is advantageous to dispense an inhalation drug with essentially no excipient, e.g., where the patient may be allergic to an excipient or the drug reacts with an excipient.

In another aspect, the invention relates to a method of administering at least one active ingredient to a patient. The method comprises providing a metered dose inhaler or metered dose inhaler system as defined herein; and activating the metered dose inhaler or metered dose inhaler system to deliver a pharmaceutically effective amount of the at least one active ingredient to the patient. Such methods may be used in the treatment of and/or the prophylaxis of a respiratory condition. For the purposes of the invention, the term “respiratory condition” encompasses, without limitation, diseases and disorders associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis).

It will be apparent to those skilled in the art that modifications to the invention described herein can readily be made without departing from the spirit of the invention. Protection is sought for all the subject matter described herein including any such modifications.

The following non-limitative Examples serve to illustrate the invention.

### EXAMPLES

### Example 1

0.6 mm wall 12.5 mL MDI cans (Presspart Inc., Cary, N.C.) were spray-coated (Livingstone Coatings, Charlotte, N.C.) with primer (DuPont 851-204) and cured to the vendor's standard procedure, then further spray-coated with either FEP or PFA (DuPont 856-200 and 857-200, respectively) and cured according to the vendor's standard procedure. The thickness of the coating is approximately 10 μm to 50 μm. These cans are then purged of air (see PCT application number WO94/22722 (PCT/EP94/00921)), the valves crimped in place, and a suspension of about 29 mg albuterol sulfate in about 18.2 gm P134a is filled through the valve.

### Example 2

0.7 mm thick aluminum sheet (United Aluminum) was spray-coated (DuPont, Wilmington, Del.) with FEP (DuPont 856-200) and cured. The thickness of the coating is approximately 10 μm to 50 μm. This sheet was then deep-drawn into cans (Presspart Inc., Cary, N.C.). These cans are then purged of air, the valves crimped in place, and a suspension of about 12 mg albuterol sulfate in about 7.5 gm P134A is filled through the valve.

### Example 3

0.6 mm 12.5 ml MDI cans (Presspart Inc., Cary, N.C.) are spray-coated with PTFE-PES blend (DuPont) as a single coat and cured according to the vendor's standard procedure. The thickness of the coating is between approximately 1 μm and approximately 20 μm. These cans are then purged of air, the valves crimped in place, and a suspension of about 31.8 mg or about 15.4 mg micronised albuterol sulphate in about 19.8 g or about 9.6 g respectively P134a is filled through the valve.

### Example 4

0.6 mm 12.5 ml MDI cans (Presspart Inc., Cary, N.C.) are spray-coated with PTFE-FEP-polyamideimide blend (DuPont) and cured according to the vendor's standard procedure. The thickness of the coating is between approximately 1 μm and approximately 20 μm. These cans are then purged of air, the valves crimped in place, and a suspension of about 31.8 mg or about 15.4 mg micronised albuterol sulphate in about 19.8 g or about 9.6 g respectively P134a is filled through the valve.

### Example 5

0.6 mm 12.5 ml MDI cans (Presspart Inc., Cary, N.C.) are spray-coated with FEP powder (DuPont FEP 532) using an electrostatic gun. The thickness of the coating is between approximately 1 μm and approximately 20 μm. These cans are then purged of air, the valves crimped in place, and a suspension of about 31.8 mg or about 15.4 mg micronised albuterol sulphate in about 19.8 g or about 9.6 g respectively P134a is filled through the valve.

### Example 6

0.7 mm thick aluminium sheet (United Aluminium) is spray coated with FEP-Benzoguanamine and cured. This sheet is then deep-drawn into cans. These cans are then purged of air, the valves crimped in place, and a suspension of about 31.8 mg or about 15.4 mg micronised albuterol sulphate in about 19.8 g or about 9.6 g respectively P134a is filled through the valve.

### Example 7

0.6 mm 12.5 ml MDI cans (Presspart Inc., Cary, N.C.) are spray-coated with an aqueous dispersion of PFA (Hoechst PFA-6900n) and cured. The thickness of the coating is between approximately 1 μm and approximately 20 μm. These cans are then purged of air, the valves crimped in place, and a suspension of about 31.8 mg or about 15.4 mg micronised albuterol sulphate in about 19.8 g or about 9.6 g respectively P134a is filled through the valve.

### Example 8

0.7 mm 12.5 ml MDI cans (Presspart Inc., Cary, N.C.) are spray-coated with PTFE-PES blend (DuPont) as a single coat and cured according to the vendor's standard procedure. The thickness of the coating is between approximately 1 μm and approximately 20 μm. These cans are then purged of air, the valves crimped in place, and a suspension of about 28.9 mg micronised albuterol sulphate in about 18 g P134a is filled through the valve.

### Example 9

0.70 mm 12.5 ml MDI cans (Presspart Inc., Cary, N.C.) are spray-coated with PTFE-FEP-polyamideimide blend (DuPont) and cured according to the vendor's standard procedure. The thickness of the coating is between approximately 1 μm and approximately 20 μm. These cans are then purged of air, the valves crimped in place, and a suspension of about 28.9 mg micronised albuterol sulphate in about 18 g P134a is filled through the valve.

### Example 10

0.70 mm 12.5 ml MDI cans (Presspart Inc., Cary, N.C.) are spray-coated with FEP powder (DuPont FEP 532) using an electrostatic gun. The thickness of the coating is between approximately 1 μm and approximately 20 μm. These cans are then purged of air, the valves crimped in place, and a suspension of about 28.9 mg micronised albuterol sulphate in about 18 g P134a is filled through the valve.

### Example 11

0.60 mm thick aluminium sheet (United Aluminium) is spray coated with FEP-Benzoguanamine and cured. This sheet is then deep-drawn into cans. These cans are then purged of air, the valves crimped in place, and a suspension of about 28.9 mg micronised albuterol sulphate in about 18 g P134a is filled through the valve.

### Example 12

0.7 mm 12.5 ml MDI cans (Presspart Inc., Cary, N.C.) are spray-coated with an aqueous dispersion of PFA (Hoechst PFA-6900n) and cured. The thickness of the coating is between approximately 1 μm and approximately 20 μm. These cans are then purged of air, the valves crimped in place, and a suspension of about 28.9 mg micronised albuterol sulphate in about 18 g P134a is filled through the valve.

### Examples 13 to 17

Examples 3 to 7 were repeated except that a suspension of 29 mg micronised albuterol sulphate in about 21.4 g P227 is filled through the valve.

### Examples 18 to 22

Examples 3 to 7 are repeated except that 66 mg, or 6.6 mg micronised fluticasone proprionate in about 182 mg ethanol and about 18.2 g P134a is filled through the valve.

Dose delivery from the MDIs tested under simulated use conditions is found to be constant, compared to control MDIs filled into uncoated cans which exhibit a significant decrease in dose delivered through use.

Experimental Data

12.5 mL aluminium canisters of the required thickness were annealed at 390° C. for 10 minutes. After cooling these canisters and a control canister (standard 12.5 mL canister 0.46-0.48 mm thickness) were subjected to the following testing.

Method Used for Compression Yield Strength.

Using a Lloyd LB000r Tensile Tester set on compression mode each sample canister was placed under the crosshead of the tester and the crosshead lowered until the canister was compressed by 4 mm in comparison to its original height.

Method for Testing Dent Resistance

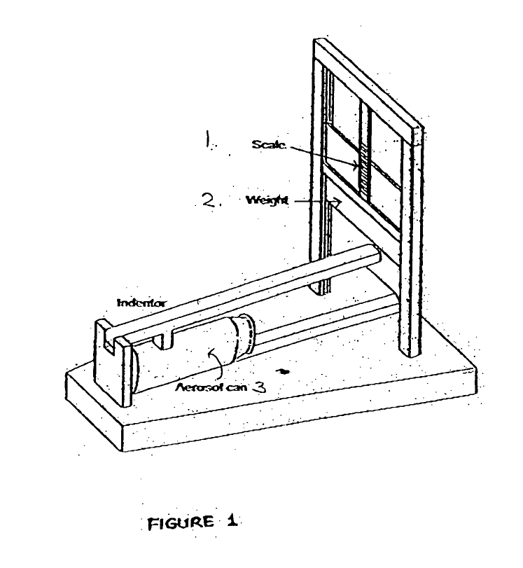

A dent resistance meter (an indentor) was constructed to measure the indentation on the wall of an aluminium canister when a weight attached to a lever arm of the indentor was dropped on it. The indentor is shown in FIG. 1.

The position on the wall of the canister where the dent was inflicted was the same for all samples tested.

Results

TABLE 1

| Canister Thickness | Compression Yield | |
|---|---|---|
| (mm) | Strength (N) | Depth of Indentation |
| Control (unannealed) | 1591 | 6 |
| 0.48 | | |
| 0.48 | 686 | 14 |
| 0.60 | 1911 | 7.5 |
| 0.70 | 2777 | 5.4 |
| 1.0 | 3104 | 4.5 |

Conclusion

The 0.48 mm annealed canister performs significantly worse than the control canister, which is unannealed. The 0.60 mm canister performs comparably to the control and the 0.70 mm canister performs better than the control, especially in respect of the compression yield strength. The 1 mm canister preforms only marginally better than the 0.70 mm canister,

We claim:

1. A canister for a metered dose inhaler having part or all of its internal surfaces coated with one or more fluorocarbon polymers, optionally in combination with one or more non-fluorocarbon polymers, said canister having a wall with a thickness in the range 0.55 mm to 1.00 mm.

2. A canister according to claim 1, wherein the wall is 0.55 to 0.70 mm in thickness.

3. A canister according to claim 2, wherein the wall is 0.60 mm in thickness.

4. A canister according to claim 1, wherein said fluorocarbon polymer is selected from PTFE, PFA, FEP and mixtures thereof.

5. A canister according to claim 1, wherein said fluorocarbon polymer is in combination with a non-fluorocarbon polymer selected from polyamideimide and polyethersulphone.

6. A metered dose inhaler comprising a canister according to claim 1.

7. A metered dose inhaler according to claim 6, containing a pharmaceutical aerosol formulation.

8. An inhaler according to claim 6, wherein said formulation comprises fluticasone propionate or a physiologically acceptable solvate thereof in combination with a bronchodilator or an antiallergic.

9. An inhaler according to claim 8, wherein said drug formulation comprises fluticasone propionate in combination with salmeterol xinafoate.

10. An inhaler according to claim 9, wherein said formulation consists essentially of fluticasone propionate or a physiologically acceptable solvate thereof, optionally in combination with one or more other pharmacologically active agents, and a fluorocarbon propellant.

11. An inhaler according to claim 6, wherein the fluorocarbon propellant is 1,1,1,2-tetrafluoroethane, or 1,1,1,2,3,3,3-heptafluoro-n-propane or mixtures thereof.

12. An inhaler according to claim 11, wherein the fluorocarbon propellant is 1,1,1,2-tetrafluoroethane.

13. A metered dose inhaler system comprising a metered dose inhaler according to claim 6 fitted into suitable channelling device for oral or nasal inhalation of the drug formulation.

14. A metered dose inhaler system comprising:
a canister for a metered dose inhaler having part or all of its internal surfaces coated with one or more fluorocarbon polymers, optionally in combination with one or more non-fluorocarbon polymers, said canister having a wall with a thickness in the range 0.55 mm to 1.00 mm, said canister including a mouth;
a crimped cap covering the mouth of the canister;
a drug metering valve situated in the cap; and
a channelling device;
wherein the inhaler system includes a pharmaceutical aerosol formulation therein, said pharmaceutical aerosol formulation comprising drug optionally in combination with one or more pharmacologically active agents.

15. A metered dose inhaler system according to claim 14 wherein said formulation comprises fluticasone propionate.

16. A metered dose inhaler system according to claim 14 wherein said formulation comprises albuterol sulphate.

17. A metered dose inhaler system according to claim 14, wherein said formulation comprises fluticasone propionate or a physiologically acceptable solvate thereof in combination with a bronchodilator or an antiallergic.

18. A metered dose inhaler system according to claim 14, wherein said drug formulation comprises fluticasone propionate in combination with salmeterol xinafoate.

19. A metered dose inhaler system according to claim 14, wherein said formulation consists essentially of fluticasone propionate or a physiologically acceptable solvate thereof, optionally in combination with one or more other pharmacologically active agents, and a fluorocarbon propellant.

20. A metered dose inhaler system according to claim 19, wherein the fluorocarbon propellant is 1,1,1,2-tetrafluoroethane, or 1,1,1,2,3,3,3-heptafluoro-n-propane or mixtures thereof.

21. A metered dose inhaler system according to claim 19, wherein the fluorocarbon propellant is 1,1,1,2-tetrafluoroethane.

22. A method of administering at least one active ingredient to a patient comprising:
providing a metered dose inhaler as defined by claim 6; and
activating the metered dose inhaler to deliver a pharmaceutically effective amount of the at least one active ingredient to the patient.
